(19) United States
(12) Patent Application Publication
(10) Pub. No.: US 2003/0180344 A1
(43) Pub. Date: Sep. 25, 2003

(54) Bioresorbable osteoconductive compositions for bone regeneration

(75) Inventors: Donald L. Wise, Belmont, MA (US); Debra J. Trantolo, Princeton, MA (US); Kai-Uwe Lewandrowski, Brookline, MA (US); Joseph D. Gresser, Brookline, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Cambridge Scientific, Inc.

(21) Appl. No.: 10/359,445

(22) Filed: Feb. 5, 2003

Related U.S. Application Data
(60) Provisional application No. 60/354,833, filed on Feb. 5, 2002.

(51) Int. Cl. A61F 2/00

(52) U.S. Cl. 424/423

(57) ABSTRACT

Bioresorbable osteoconductive compositions and methods of using the composition as a scaffold for bone repair in periodontal, alveolar or maxillary regeneration, bony cranial defects, and spinal regeneration are disclosed. The bioresorbable compositions contain a bioresorbable polymer, a micro or nano particulate filler and a pore creating substance. The bioresorbable polymer can be electronically unsaturated and cross-linkable with a cross-linking agent. The micro or nano filler can be any natural biocompatible material such as a metals, calcium carbonate, carbon, a biocompatible synthetic material, or a bioceramics such as hydroxyapatite. The pore creating substance can be an effervescent agent such as a carbonate and an acid.

This claims priority to U.S. S. No. 60/354,833 filed Feb. 5, 2002.

The United States government has rights in this invention by virtue of NIH/NIDR Grant No. 1 R43 DE 12290-01A1 to Joseph D. Gresser, and NIH/NIAMS Grant AR 45062 to Kai-Uwe Lewandrowski).

### BACKGROUND OF THE INVENTION

The present application relates generally to using bioresorbable osteoconductive compositions, and the scaffolds formed therefrom, for bone repair. In a preferred embodiment, the present application relates to methods of using bioresorbable osteoconductive compositions containing micro or nano fillers and pore forming agents for oral reconstruction such as periodontal, alveolar, or maxillary regeneration, repair of bony cranial defects and spinal repair.

A study in the mid-1980's estimated that about four and a half million people suffer fractures each year in the United States alone. Adding to the problem are bone diseases such as periodontal diseases which result in bone loss. Bone repair materials, therefore, are actively sought for bone repair and regeneration. Biodegradable and biocompatible polymeric compositions are useful for bone grafting, bone repairing, bone replacement, or bone-implant fixation purposes.

Many problems exist with the present bone grafting, bone repairing, and bone-implant fixation methods. In periodontal reconstruction or implant fixation, bony voids need to be filled with a graft material that supports the structural integrity of the site throughout the course of new bone regeneration. Presently available modalities for the treatment of periodontal diseases with subsequent bone loss ameliorate their progression, but have only minimal potential to regenerate the supporting apparatus of the tooth.

This is also true in maxillofacial and mandibular reconstruction. Autografts and allografts are used in current bone graft procedures. Autografts are preferable, but are not always available in sufficient quantities or may not produce a clinically desired result. Bone replacement materials for maxillofacial, alveolar and mandibular reconstruction are in use as alternatives to autografts. Clinically applied techniques include the use of biodegradable membranes for guided tissue regeneration during bony recovery after grafting procedures. However, despite significant advances in the development of these technologies to better approximate the three-dimensional nature of complex tissue equivalents, the development of clinically applicable bone replacement materials has remained a challenge. At least in part, the challenge lies with the difficulty in enabling sufficient ingrowth of repair tissues into biodegradable repair materials for prolonged periods of time so that the bony architecture at the defect site is preserved. Implantation of such materials in skeletal repair sites commonly produces on-growth that is often limited to the periphery of the implant rather than a through-and-through tissue penetration. The latter process, however, appears eminently important for the successful development and manufacturing of universal tissue equivalents for maxillofacial and periodontal applications.

In light of the drawbacks to currently approved synthetic products, including a lack of resorbability, inclusion of animal or marine derived components, and poor handling characteristics, the goal becomes to create a bone repair material, which behaves both biologically and biomechanically, more like the maxillofacial bones and the mandible.

Bioceramic fillers have been used to provide mechanical strength and structural integrity of bone reconstruction materials. For example, Ca₁₀(PO₄)₆(OH)₂, hydroxyapatite (HA), is a widely-utilized bioceramic material for bone repair because it closely resembles native tooth and bone crystal structure. For example, U.S. Pat. No. 5,425,769 to Snyders describes an artificial bone substitute composition consisting of fibrous collagen in a calcium sulfate matrix.

Nonetheless, conventional materials lack compositional purity and homogeneity and, thus, are unable to provide desired structural integrity. Attempts to solve the problem only give limited success. For example, Jarcho, et al. Science 11, 2027-2035 (1976) describes a process for forming dense polycrystalline HA that is “substantially stronger than other HA materials”, and that elicits “an excellent biological response when implanted in bone”. A precipitation method was used and material of average grain size of 150-700 nm recovered. However, Jarcho et al. (1976) reported low volume fraction of pores and reported considerable grain growth during sintering, even at firing temperatures of 1000° C. Jarcho et al. (1976) achieved 99% density in some cases, but used a technique that can be impractical for forming desired shapes. Akao et al. J. Mater. Sci. 16, 809-812 (1981) reported the compressive flexural and dynamic torsional strengths of polycrystalline HA sintered at 1300° C. for three hours and compared the mechanical properties of the product with those of cortical bone, dentine, and enamel. The compressive strength of the sintered HA was approximately 3-6 times as strong as that of cortical bone. Hench J. Am. Ceram. Soc. (1991) reported that HA had been used as a coating for porous metal surfaces for fixation of orthopaedic prostheses, in particular, that HA powder in the pores of porous, coated-metal implants would significantly affect the rate and vitality of bone ingrowth into the pores. It is reported that many investigators have explored this technique, with plasma spray coating of implants generally being preferred. Hench (1991) reported, however, that long-term animal studies and clinical trials of load-bearing dental and orthopaedic prostheses suggest that the HA coatings may degrade or delaminate. Therefore, their applications as fillers for bone regeneration are limited.

Conventional biocompatible materials are hard to fabricate meanwhile maintaining their mechanical as well as structural integrity. For example, HA is difficult to sinter. As such, dense HA structures for dental implants and low-wear, orthopaedic applications typically have been obtained by high-temperature and/or high-pressure sintering with glassy sintering aids, which frequently induce decomposition to undesirable phases with poor mechanical stability and poor chemical resistance to physiological conditions.

Hydroxyapatite has been used in a number of applications. For example, U.S. Pat. No. 6,241,771 describes a resorbable interbody fusion device for use in spinal fixation. The device is composed of 25-100% bioresorbable or resorbable material and a neutralization compound, or buffer, which is hydroxyapatite. EPA 99942186.0 by Cambridge Scientific, Inc. describes a biocompatible tissue transplant formed of a solid biocompatible substrate formed into a suitable shape having a porous coating thereon formed of a biocompatible biodegradable polymer wherein cells capable of regenerating autologous tissue are seeded onto the surface of the polymer coating. EPA 99966346.1 by Cambridge Scientific, Inc. describes a method of producing a perforated and partially demineralized cortical bone allograft for transplatation, by filling the perforations in the perforated and partially demineralized cortical bone allograft with a biodegradable porous polymeric matrix.

It is therefore an object of the present invention to provide bioresorbable compositions or scaffolds with fillers of enhanced structural integrity for bone reconstruction.

It is another object of the present invention to provide bioresorbable compositions or scaffolds with fillers of enhanced structural integrity for bone grafting or bone tissue regeneration.

It is another object of the present invention to provide bioresorbable compositions or scaffolds with fillers of enhanced structural integrity for periodontal, alveolar or maxillary regeneration.

### SUMMARY OF THE INVENTION

Bioresorbable osteoconductive compositions and the methods of using such compositions for bone reconstruction are disclosed. The composition contains a bioresorbable polymer, a micro or nano biocompatible filler, and, preferably, a pore creating substance. The bioresorbable polymer can be electronically unsaturated and cross-linkable with a cross-linking monomer. The micro or nano filler can be any biocompatible material such as a biocompatible metal, calcium carbonate, a biocompatible synthetic material, carbon, or a bioceramic such as hydroxyapatite (“HA”). The pore creating substance can be an effervescent agent such as a carbonate and an acid, such as sodium bicarbonate and the acid can be citric acid. In one preferred embodiment, the bioresorbable polymer is polypropylene glycol-fumaric acid. In another preferred embodiment, the monomer is vinyl pyrrolidone. In still another preferred embodiment, the micro or nano filler is HA.

The compositions can be used for bone tissue regeneration, for example, for oral reconstruction. Generally, the oral reconstruction relates to repair of a mandibular or maxillofacial defect, maxillofacial bone grafting such as sinus augmentations and onlay graft, or ridge expansion. In another embodiment, the oral reconstruction is periodontal, alveolar or maxillary regeneration. In a specifically preferred embodiment, the oral reconstruction is tooth replacement. The compositions can have other uses such as spinal segment repair, repair of bony cranial defects and as bone graft extenders. Methods of bone repair or grafting generally involve first fabricating an appropriate template formed of the composition and then implanting the article in a mammal in need of bone repair.

### BRIEF DESCRIPTION OF THE DRAWING

FIG. 1 summarizes failure load and stiffness results normalized with respect to the intact motion segment during axial compression.

FIG. 3. CSI-02. Mandible Defect Area.

Area for the untreated defect and defect receiving demineralized bone from the same mandible was calculated from the mean of the x and y axis using the area of a circle equation (πr²). Group means and standard errors of the mean (SEM) were calculated for each week. The plot shows the defect area for untreated and defect receiving demineralized bone.

### DETAILED DESCRIPTION OF THE INVENTION

I. Bioresorbable Polymeric Compositions with Micro or Nano Fillers

A. Bioresorbable Polymeric Materials

Polymers must be non-toxic, biodegradable, and/or bioresorbable, i.e., their degradation products are used by or are otherwise eliminated from the human body via existing biochemical pathways. The preferred biocompatible polymers are polyesters or other hydrolytically degradable polymers. The polyesters can be chemically cross-linkable, i.e., possess functional groups which will allow the polyester polymer chains to be reacted with cross-linking agents-reactive with said functional groups. Suitable polyester materials include polyesters formed from biocompatible di- and tri-carboxylic acids or their ester-forming derivatives (e.g., acid chlorides or anhydrides) and di- or polyhydric C₂-C₆ alcohols. The functional groups in the polyester allowing for polyester cross-linking can derive from either the alcohol or the acid monomer components of the polyester.

Representative carboxylic acids for formation of polyesters include Kreb's cycle intermediates such as citric, isocitric, cis-aconitic, alpha-ketoglutaric, succinic, malic, oxaloacetic and fumaric acid. Many such carboxylic acids have additional functionalities which can allow cross-linking and therefore means for curing the bioresorbable compositions from a paste-like moldable mass to a hardened cement state. Fumaric acid is a preferred acid for forming the polyester. It is a dicarboxylic acid having a free-radical reactive double bond well suited for free radical induced cross-linking reactions. Illustrative of C₂-C₆ alkyl or aklylene alcohols useful to form polyesters are ethylene glycol, 2-buten-1,4-diol, 2-methyl-2-buten-1,4-diol, 1,3-propylene glycol, 1,2-propylene glycol, glycerine, 1,3-butanediol, 1,2-butanediol, 4-methyl-1,2-butanediol, 2-methyl-1,3-propanediol, 4-methyl-1,2-pentanediol, cyclohexen-3,4-diol and the like. In a preferred embodiment the polyester component of the bioresorbable compositions is poly(propylene glycol fumarate) (PPF) formed by the condensation (esterification) reaction of propylene glycol and fumaric acid.

PPF is advantageous because PPF possesses two chemical properties that are critical to the function of a biodegradable bone cement. The first is the ease by which PPF can be degraded in vivo into its original fumaric acid and propylene glycol subunits. Both fumaric acid and propylene glycol are non-toxic and well-tolerated in vivo. As a Kreb's cycle intermediate, fumaric acid plays an essential role in the process by which food is converted into energy. Propylene glycol is used throughout the food industry as a food additive and can be metabolized or excreted by the body. The second critical property is that each subunit of the PPF prepolymer contains an activated unsaturated site through which the polyester can be cross-linked with various olefinic free-radical induced cross-linking agents.

The polyester may be cross-linked during the curing period. Where the reactive chemically functional groups in the polyester are carbon-carbon double bonds (e.g., in the preferred PPF polyester component) representative cross-linking agents are N-vinylpyrrolidone (VP), methyl methacrylate (MMA), and like olefinic cross-linking agents. A preferred cross-linking agent is MMA, which exists as a clear liquid at room temperature. It is particularly suitable for free radical induced cross-linking of PPF in accordance with a preferred embodiment of this invention.

Other examples of useful polymers include alphahydroxy acids such as poly(L-lactic acid), poly(D L-lactic acid), poly(D L-lactic-co-glycolic acid), and poly(glycolic acid), poly(epsilon-caprolactone), polyorthoesters, polyanhydrides, polydioxanone, copoly(ether-esters), polyamides polylactones and combinations thereof. These polymers may be obtained in or prepared with the molecular weights and molecular weight distribution needed for a desired use. Suitable solvent systems for preparation of these polymers are published in standard textbooks and publications. See, for example, Lange's Handbook of Chemistry, Thirteenth Edition, John A. Dean, (Ed.), McGraw-Hill Book Co., New York, 1985. These polymers may be formed into fibers and webs by standard processing techniques including melt extrusion and spin casting, and are commercially available in woven or non-woven form.

Bioresorbable polymers are known, commercially available, or can be synthesized using known and published methods. Bioresorbable polymers have been described for a variety of applications, including controlled release dosage forms and bioresorbable sutures. See U.S. Pat. Nos. 3,463,158; 4,080,969; 3,997,512; 4,181,983; 4,481,353; and 4,452,973. Ibay et al. Polym. Mat. Sci. Eng. 53, 505-509 (1985) describe the preparation and use of moldable implant appliances from vinylpyrrolidone cross-linked poly(propylene glycol fumarate) (PPF) for use as temporary replacements for soft tissue and/or bone following trauma. Absorbable polyglycolic acid suture has been used successfully for internal fixation of fractures. B. Roed-Peterson, Int. J. Oral. Surg., 3, pp. 133-136 (1974).

U.S. Pat. No. 5,522,895 to Mikos described biodegradable bone templates formed of biodegradable polymers. Useful biodegradable materials are, for example, poly(L-lactic acid), poly(D,L-lactic acid), poly(D,L-lactic-co-glycolic acid), poly(glycolic acid), poly(epsilon-caprolactone), polyortho esters, and polyanhydrides, which have the capacity of being rendered porous. Gerhart, et al., used biodegradable polyesters that are chemically crosslinkable with cross-linking agents to form bone cements (U.S. Pat. No. 4,843,112 to Gerhart, et al.). Other illustrative examples of application of bioresorbable or biodegradable polymers are described in U.S. Pat. No. 6,071,982 to Wise et al.; U.S. Pat. No. 4,888,413 to Domb; U.S. Pat. No. 5,733,951 to Yaszemski et al.; and U.S. Pat. No. 4,722,948 to Sanderson.

B. Pore-Forming Agents

Porosity of a bone cell carrier facilitates bone cell growth. For example, U.S. Pat. No. 5,522,895 to Mikos describes a bioresorbable, three-dimensional template for repair and replacement of diseased or injured bone that provides mechanical support to bone while providing a guide for growth of bone tissue. The template is formed of biodegradable materials in the form of a continuous matrix and a pore-forming component having a rate of degradation which exceeds that of the matrix. Differential dissolution or biodegradation provides porosity to the template. Similarly, U.S. Pat. No. 4,722,948 to Sanderson reported a bone replacement and repair material prepared from a biocompatible polyester resin, a liquid linking agent capable of cross-linking the resin and a filler which is moldable and formable and cures in vivo. The resulting cured putty degrades in vivo to provide interstices in the polyester matrix for new tissue growth.

Pores contained in the bioresorbable polymeric compositions can be in any form. In one embodiment, pores of the bioresorbable compositions can be generated via adding to the bioresorbable composition a biodegradable material in the form of fibers or webs having a faster biodegradation rate than that of the bioresorbable polymer. In another example, particles of biocompatible and water soluble organic or inorganic material such as sugar and starch or organic or inorganic salts such as NaCl and KCl can be used to generate the pores. These materials are incorporated into the composition, the composition solidified, and the particulates extracted using a water extraction technique. Alternative, the particles can be formed of a volatile salt, which is removed by application of a vacuum or lyophilization. In still another embodiment, an effervescent agent can be used to generate the pores in the form of foam. In an especifically preferred embodiment, the foam is formed by including effervescent fillers that generate CO₂ as the material cures. In the most preferred embodiment, citric acid and a carbonate or bicarbonate such as sodium bicarbonate are used to form the effervescent agent.

The pore size can be controlled by varying the ratio of the effervescent agent to the polymeric material and varying the particle size of the effervescent agent. To facilitate osteoblast migration, a polymer matrix or foam with pore sizes of 100-300 microns is desirable. In one embodiment wherein sodium bicarbonate (SB) and citric acid (CA) are used as an effervescent agent, the design of the porosity of a foam is attainable by control of the SB and CA content and by control of the sizes of the SB/CA particles used in the effervescent filler. The reaction of CA/SB with water produces carbon dioxide, the blowing agent responsible for foam formation and expansion. The stoichiometry requires a mole ratio of CA:SB in the range from 0.2:1 to 1:5, preferably 1:3. The moles of CO₂, which can be generated per gram of material, depend on the loading of CA/SB in the foaming cement. For example, a 0.15% CA/SB loading would produce a 25% expansion at 37° C. and 1 atm based on the above stoichiometry.

C. Micro and Nano Fillers

Micro and/or nanocrystalline or nanocomposite materials are incorporated into the polymeric material. Any biocompatible micro or nano materials can be used as fillers in the compositions and/or scaffolds disclosed herein. Exemplary micro or nano materials are biocompatible metals, calcium carbonate, carbon, biocompatible synthetic polymeric materials, and bioceramics. In one embodiment, the micro or nano material is a bioceramic material such as HA. In another embodiment, the micro or nano material is a biocompatible metal such as nickel (Ni), titanium (Ti), aluminum (Ai), gold (Au), platinum (Pt), iron (Fe), silver (Ag), Copper (Cu). By designing materials from the cluster level, crystallite building blocks of less than 10 nm can be made, through which unique size-dependent properties such as quantum confinement effect and superparamagnetism can be obtained.

Methods of forming various nano particle materials are well known. Various nanocrystalline ceramics for structural applications were rigorously investigated in the 1990's. Siegel, “Recent progress in nanophase materials”, in B Minerals, Metals and Materials, Suryanarayana, et al., ed (1996) discussed nanophase metals and ceramics noting that while many methods exist for the synthesis of nanostructured materials, including chemical or physical vapor deposition, gas condensation, chemical precipitation, aerosol reactions, and biological templating, synthesis and processing methods for creating tailored nanostructures are sorely needed, especially techniques that allow careful control of surface and interface chemistry that can lead to adherent surface coatings or well-consolidated bulk materials. In the case of normally soft metals decreasing grain sizes of the metal below a critical length scale (less than about 50 nm) for the sources of dislocations in the metal, increases the metal's strength. Clusters of metals, intermetallic compounds, and ceramics have been consolidated to form ultrafine-grained polycrystals that have mechanical properties remarkably different and improved relative to their conventional coarse-grained counterpart. Nanophase copper and palladium, assembled from clusters with diameters in the range of 5-7 nm, are noted for having hardness and yield strength up to 500% greater than in conventionally-produced metal. It is also noted that ceramics and conventionally brittle intermetallics can be rendered ductile by being synthesized from clusters with sizes below 15 nm, the ductility resulting from the increased ease with which the ultrafine grains can slide by one another in “grain-boundary sliding.” U.S. Pat. No. 5,130,277 to Zettle et al., described a method and an apparatus for making nanotubes and nanoparticles. A method for forming nano-sized powders of alpha alumna from a boehmite gel doped with a barrier-forming material such as silica that is then dried, fired and comminuted to powder form was reported by U.S. Pat. No. 6,048,577 to Garg.

The micro or nano materials generally have a particle size in the range of less than 1000 microns, more preferably less than 100 microns, even more preferably less than 10 micron, or in the nanoparticle range. As demonstrated using hydroxyapatite micro- and nano-particles, there is more bone formation with implants containing nanoparticular HA. Nano-HA is more homogeneous and of higher purity than conventional HA. It also has better mechanical properties.

D. Biologically Active Materials

Biologically active materials include pharmaceutically active materials as well as cells can be incorporated into the composition. Pharmaceutically active materials such as therapeutic agents like growth factors and/or other drugs or agents can be used to enhance bone regeneration and/or tissue adhesion (See Lowenguth and Blieden, 1993, Periodontology 2000, 1:54-68). There are numerous examples using growth factors or other pharmaceutically active materials in bone regeneration. Illustrative examples are U.S. Pat. Nos. 4,861,757, 5,019,559, and 5,124,316 to Antonaides et al, using purified growth factors; U.S. Pat. No. 5,149,691 to Rutherford, using growth factors in combination with dexamethasone to enhance the mitogenic effect of the growth factor; U.S. patent No. Terranova et al., using root surface demineralization; and U.S. Pat. No. 4,916,707 using periodontal barriers such as membranes. In another example, periodontal barriers have been designed so that they may also be used for the controlled delivery of chemotherapeutic agents such as tissue regenerative agents like growth factors, antibiotics, and antiinflammatory agents to promote periodontal healing and regeneration.

Therapeutic agents may be incorporated for timed release of these agents in situ. For example, agents may be incorporated into the polymeric matrix or the pore-creating substance, and are slowly released as the matrix is degraded. Growth factors, particularly platelet-derived growth factors (PDGF) and insulin-like growth factor (IGF-1) are known to stimulate mitogenic, chemotactic and proliferative (differentiation) cellular responses. Preferred pharmaceutically active materials are those that enhance bone regeneration and/or tissue adhesion. Illustrative examples include growth factors, antibiotics, immunostimulators, and immunosuppressants. In one embodiment, the pharmaceutically active material is a bone repair protein such as BMP. In another embodiment, the pharmaceutically active material is a growth factor such as FGF or agent which promotes the generation of connective tissue.

Cells can also be incorporated on or in the matrix. Bone cells can grow in synthetic polymeric as well as in natural matrixes (Uchida et al., Acta. Orthoapedica Scand. 59, 29-33 (1988). Bone cells taken from a future recipient can be expanded in vitro by engineering for use in bone repair. For example, Breitbart et al., Plast. Reconst. Surg. 101(3), 567-574 (1998) demonstrated the feasibility of using periosteal cells for tissue engineered bone repair of calvarial defects. Ishaug et al. J. Biomed. Mater. Res. 28, 1445-1453 (1994); Biotechnol. Bioengin. 50, 443-451 (1996) demonstrated that osteoblasts can grow and migrate throughout polymeric scaffolds in vitro. Ishaug-Riley et al. Biomaterials 19, 1405-1412 (1998) then demonstrated that osteoblast function on synthetic materials was equal to that of nondegradable orthopaedic materials. Seeded resorbable scaffolds are replaced by new bone when implanted into bony sites.

In one embodiment, the compositions or scaffolds are PPF-based compositions or scaffolds which present sufficient hydrophilicity for cell attachment and proliferation. When used along with proper fillers, the PPF-based compositions or scaffolds offer demonstrable porosity for cellular migration, generate a richness of surface area for neo-vascularization, and provide sufficient dimensional stability for support of the reconstructive process.

II. Preparation of Bioresorbable Compositions with Micro or Nano Fillers

The preparation of the bioresorbable compositions disclosed herein typically involves combining the polymer and the cross-linking agent into a substantially homogeneous mixture to form a moldable composite cement mass which hardens on curing, i.e., completion of the cross-linking reaction. The number average molecular weight [M(n)] and molecular weight distribution [MWD] of the polymer should be such that the polymer and cross-linking agent can be combined to form a substantially homogeneous mixture. Preferably the cross-linking agent is a liquid and the polymer is substantially soluble in, or miscible with, the cross-linking agent. Alternatively, the cross-linking agent can be a solid soluble in a liquid low molecular weight polymer, or a liquid miscible therewith. Under ideal circumstances, the cross-linking reaction will result in a homogeneous (uniformly cross-linked) polymer/particulate composite cement.

In a preferred embodiment, poly(propylene glycol fumarate)(PPF) is combined with an amount of methyl methacrylate sufficient upon reaction initiation to cross-link the polyester to the level necessary to form a rigid cross-linked PPF polymer matrix for admixed particulate calcium salts. Preferred MWD for the PPF ranges from about 500 to about 1200 M(n) and from about 1500 to about 4200 M(w). In a preferred embodiment, the liquid polymer phase of the bioresorbable compositions is about 80 to about 95 percent by weight PPF and about 5 to about 20 Percent by weight MMA monomer. The optimal weight percentages for mechanical strength are approximately 85 percent PPF and about 15 percent MMA. The MMA monomer is typically stabilized to prevent premature polymerization, i.e., prior to mixing with PPF, with a few parts per million of hydroquinone.

It is important that the proportions of polymeric composition and the cross-linking agent are controlled. For example, if too much MMA monomer is added, the MMA molecules can polymerize themselves without being interrupted by the PPF chains. The result is a material that behaves like conventional PMMA bone cement and does not biodegrade. If too little VP monomer is added, the PPF polymer chains will not be effectively cross-linked and the cement will not cure to form a matrix of sufficient rigidity. However, the knowledge of how much cross-linking agent to use with respect to a particular cross-linking agent is within the skill in the art which can be readily determined without undue experimentation.

The VP-PPF cross-linking reaction proceeds via a free-radical propagated polymerization reaction. The cross-linking reaction therefore is, in practice, accelerated by addition of a free-radical initiator. One suitable free-radical initiator for this process is benzoyl peroxide. Other peroxides such as t-butyl hydroperoxide and methyl ethyl ketone peroxide and other free-radical initiator such as t-butyl perbenzoate are also suitable free-radical initiator for this process.

A catalytic amount (less than 1% by weight) of dimethyltoluidene (DMT) is typically added to accelerate the formation of free radicals at room temperature. Thus, the rate of cross-linking (i.e. time for curing or hardening of the bioresorbable compositions) can be adjusted by controlling the amount of DMT added to the PPF/MMA mixture. The curing rate can be adjusted so that the bioresorbable compositions are substantially cured in a period ranging from less than a minute to over 24 hours. The preferred curing time depends, of course, upon what is the most practical period of time for surgical purposes. The curing period should be sufficiently long to allow the surgeon time to work with the bioresorbable composition to mold it or apply it to the appropriate surfaces. At the same time, the cure rate should be high enough to effect, for example, implant stabilization within a short time following the surgical procedure. The polymerization or solidification period for bone implant fixation typically ranges from about 5 to about 20 minutes, and preferably about 10 minutes.

A number of procedures can be used to generate the porosity. In one embodiment wherein water soluble organic or inorganic salt particles are used to create the pores, the particles are leached out or otherwise removed from the matrix leaving a polymeric matrix with high porosity. In another embodiment wherein polymeric fibers or webs are dispersed within a formed polymeric matrix, the dispersed fibers and the surrounding matrix possess differential rates of degradation, with the fibers being degraded at a faster rate than the matrix, thereby being removed from the template and creating a highly porous polymeric template. In a preferred embodiment wherein an effervescent agent such as SB/CA, the pores can be generated in the form of foam upon exposure to water.

III. Use of the Compositions and Scaffolds for Bone Reconstruction

The compositions can be used as scaffolds or fixtures for regeneration of bones or tissues of any type. In one embodiment, the compositions can be used as scaffolds in periodontal tissue regeneration such as regeneration of soft tissue, cementum, or bone regeneration. In another embodiment, the compositions can be used as fixtures for bone regeneration such as regeneration of spinal segments or repair of bony cranial defects. In still another embodiment, the composition can be used as bone repair material (i.e., not to replace bone but to facilitate healing). In one preferred embodiment, the compositions can be used as a bone graft extender to enhance new bone formation when mised with allo-, auto-, or xenograft materilas.

The bone repair or regeneration can be any type of bone repair, specifically oral reconstruction, spinal segment repair, bone graft extension. In one specific embodiment, the bone repair is either periodontal, alveolar, or maxillary regeneration. One specific embodiment is wherein the bone repair is tooth replacement.

In the alternative, the method of using the bioresorbable composition can include 1) fabricating ex vivo an appropriate template in a desired shape for a desired use, then 2) implanting the template in an appropriate site of application, where the template governs the shape of the new material which is formed.

The composition described herein is useful alone or in combination with other materials such as a bone graft, in applications such as the following.

A. Clinical Management of Mandibular and Maxillofacial Defects

Bone Grafting

Bone grafting procedures have become almost an integral part of implant reconstruction. In many instances, a potential implant site in the upper or lower jaw does not offer enough bone volume or quantity to accommodate a dental implant. This is usually a result of bone resorption that has taken place because one or more teeth were lost. Bone grafting procedures usually try to re-establish bone dimension, which was lost due to resorption. Common grafting materials can be categorized into five different categories: a) autograft or autogenous bone graft, b) allograft or allogenic bone graft, c) xenograft or xenogenic bone graft, d) alloplast or alloplastic bone graft, and e) growth factors.

Autograft or autogeneous bone graft is considered the gold standard. The best success rates in bone grafting have been achieved with autografts. Form most grafting purposes confined to oral implantology, part of the jaw (i.e., chin or back portions of jaw) can be used as an acceptable donor site. Sometimes, however, when there is not enough bone volume available intraorally, iliac crest bone is harvested. This source of allograft is usually cadaver bone, which is available in large amounts. Cadaver bone has to undergo many different treatment sequences in order to render it neutral to immune reactions and to avoid cross contamination of host diseases. These treatments may include irradiation, freeze-drying, acid washing and other chemical treatments. Xenograft or xenogenic bone graft is often of bovine origin. Tissue banks usually choose this graft material because it is possible to extract larger amounts of bone with a specific microstructure, which is an important factor for bone growth as compared to bone from human origin. Alloplast or alloplastic bone graft usually includes any synthetically derived graft material not derived from animal or human origin. In oral implantology, this usually includes hydroxyapatite or any formulation thereof.

Sinus Augmentations

One of the most frequently applied grafting procedures is the sinus augmentation. This procedure is restricted to the upper jaw. With aging, the pneumatization of the para-nasal sinuses occurs. Once teeth are lost in that particular area it makes it difficult to place endosseous implants in that area. For this particular problem, grafting methods were developed to literally raise the bottom of the sinus, graft bone underneath and, thus, creat enough space for one or more dental implants.

A considerable volume of bone (5 cc to 10 cc per side) is needed to perform a typical sinus augmentation. This amount of bone is usually more than can be harvested from intraoral donor sites. Therefore, use of an allograft, alloplast or xenograft or a combination (sometimes mixed with a little autograft) is sometimes necessary. However, an autograph usually takes approximately four to six months to mature in the sinus; whereas an allograft, alloplast or xenograft may take nine months or more to mature.

Sinus augmentations and implant placement can sometimes be performed as a single procedure if enough bone between the upper jaw ridge and the bottom of the sinus is available to stabilize the implant well. If insufficient bone is available, the sinus augmentation will have to be performed first, then the graft will have to mature for several months, depending on the graft material used. Once the graft has matured, the implants can be placed. In that case, the compositions provided herein, as described foregoing, can be used in augmenting autologous bone grafting.

Onlay Grafts

Onlay grafting procedure is designed to re-establish bone, which has been lost in a particular area due to resorption which, again, has been brought on by previous tooth loss in that area. Commonly, several pieces of autogenous bone, which is usually from the chin or the very back of the lower jaw, is attached to the site with the bone deficiency. The area is then closed up and after a certain healing and maturing period, this piece of bone will eventually be incorporated into the host bed and become solidly fused, so that at a later time implants can be placed in that same area. For those cases in which larger areas of resorption will need to be augmented with more pieces of autogenous bone, the patient's bone from the iliac crest or tibia is used.

Ridge Expansion

Ridge expansion is used to restore lost bone dimension when the jaw ridge get too thin to place conventional rootform implants. In this procedure, the bony ridge of the jaw is literally expanded by mechanical means. A series of expanders, which can be in cross-section round or D-shaped metal rods of successively increasing diameter, are forced into the implant site. This is accomplished by tapping these expanders into the ridge with a surgical mallet. If done properly, the use of expanders will compress the inner spongy part of the bone and bulge out of the outer cortex. At this point, an appropriate implant can either be placed immediately into the created socket or one can place a bone graft into it first and let it mature for a few months before placing the implant.

Augmentation

Newer bone grafting materials have been tested for augmentation procedures. However, the use of osteoconductive bone substitutes in this indication is controversial. It has been postulated that their use can lead to a prolonged healing time, inhomogenous ossification, foreign body reaction, migration of particles and low bone-implant contact. To eliminate this problem, combinations of an osteoinductive protein (recombinant human osteogenic protein-1 (rhOP−1=bone morphogenetic protein-7) with natural bovine bone mineral (BioOss) have been investigated in a sinus floor augmentation with simultaneous placement of implants. Terheyden, et al., (1999), augmented the maxillary sinus floors in five miniature pigs with three mL BioOss containing 420 micrograms rhOP-1 on the test side and three mL BioOss alone on the control side. At the time of augmentation, a titanium implant (ITI) was inserted from a laterocaudal direction. After six months of healing, the percentage of bone-implant-contact was 42% higher in the augmented group. It was concluded that recombinant growth factors may be delivered by natural bone mineral.

Distraction osteogenesis has been proposed both for the closure of a wide alveolar cleft and fistula in cleft patients and for the reconstruction of maxillary dentoalveolar defects in trauma patients. The objective is to create a segment of new alveolar bone and attach gingiva for the complete approximation of a wide alveolar cleft/fistula and the reconstruction of a maxillary dentoalveolar defect. This procedure has recently been performed on one patient with a traumatic maxillary dentoalveolar defect and 10 patients with unilateral or bilateral cleft lips and palates who had varied dentoalveolar clefts/fistulas (Liou et al., Plast. Reconstr. Surg., 105(4), 1262-72, 2000). Interdental and maxillary osteotomies were performed on one side of the dental arch by the cleft or defect. After a latency period of 3 days, the osteotomized distal segment of the dental arch was then distracted and transported toward the cleft or defect by using a tooth-borne intraoral distraction device. The alveoli and gingivae on both ends of the cleft or defect were approximated after distraction osteogenesis. This method may eliminate the need for extensive alveolar bone grafting.

Dental Implant

The type of dental implant used is often dependent upon the state of the maxillofacial bone. The thickness and volume of the bone will dictate the type of implant installed. In addition, grafting and reconstruction techniques are often a necessary first step to the placement of dental implants. In general, dental implants can be categorized into three main groups: (1) endosseous implants, (2) subperiosteal implants, and (3) transosseous implants.

Endosseous implants are surgically inserted into the mandible. Subperiosteal implants typically lie on top of the mandible, but underneath the gum tissues. The important distinction is that they usually do not penetrate into the jawbone. Transosseous implants are similar in definition to endosseous implants in that they are surgically inserted into the mandible; however, they are different in their orientation. Endosseous implants are the most frequently used implants today. Examples of each implant are described below.

Ramusframe Implants

Ramusframe implants are endosseous implants. These implants are designed for the toothless lower jaw only and are surgically inserted into the jaw bone in three different areas: the left and right back area of the jaw, and the chin area in the front of the mouth. These types of implants are usually used in a severely resorbed, toothless lower jawbone, which does not offer enough bone height to accommodate rootform implants as anchoring devices. These implants are usually indicated when the jaws are resorbed to the point where subperiosteal implants are no longer sufficient. An additional advantage that comes with this type of implant is a tripodial stabilization of the lower jaw. Once surgically inserted, a bar, running from one side of the jaw to the other is visible in the mouth. A denture can then be attached to the bar. Blade implants are not frequently used, however they do find an application in areas where the residual bone ridge of the jaw is either too thin (due to resorption) to place conventional rootform implants or certain vital anatomical structures prevent conventional implants from being placed. Frequently, if a certain area of the jawbone is too thin and has undergone resorption due to tooth loss it is recommended to undergo a bone grafting procedure, which re-establishes the lost bone, so that conventional rootform implants can be placed. It is for applications such as this that the material described herein would be especially well suited.

Subperiosteal Implants

Of all currently used devices, it is the type of implant that has had the longest period of clinical application. These implants are shaped to ride on the residual bony ridge of either the upper or lower jaw. Subperiosteal implants have been used in completely edentulous as well as partially edentulous upper and lower jaws. However, the best results have been achieved in treatment of the edentulous lower jaw. Indications usually include a severely resorbed, toothless lower jawbone, which does not offer enough bone height to accommodate rootform implants as anchoring devices.

Rootform Implants

Because of their osseointegration, these titanium implants have become the most popular implants. They are regarded as the standard of care in oral implantology. These implants can be placed wherever a tooth or several teeth are missing, when enough bone is available to accommodate them. However, even if the bone volume is not sufficient to place rootform implants, bone-grafting procedures within reasonable limits should be initiated in order to benefit from these implants. Some newer implants have an outer coating of hydroxyapatite. Other implants have their surface altered through plasma spraying, aderchims or beading process. Other variations focus on the shape of the rootform implant. Some are screw-shaped, others are cylindrical, or even cone-shaped or any combination thereof.

The microparticle or nanoparticle compositions described herein can be used to support the reconstructive process by allowing (1) high density of ingrowing bone cells within the scaffold, (2) integration of the ingrowing tissue with surrounding tissue following implantation, (3) vascularization, and (4) cosmetic recovery. The method of using the compositions will vary with specific procedures pertaining to the particular desired application.

B. Periodontal Tissue Regeneration for Implant Support

The material and methods disclosed herein can be used for periodontal regeneration. Periodontal regeneration can be soft tissue, cementum or alveolar bone healing of a type characteristic of the anatomy and architecture of undiseased periodontium. Generally, periodontal regeneration involves inserting a subperiosteal implant on the dental bone ridge of a mammal. Alternatively, a therapeutically effective amount of a growth factor can be used along with the insertion of the subperiosteal implant. The inserted periodontal regenerate system may be then molded to form a periodontal barrier when used for treatment of periodontal disease around a root of a tooth. The barrier can be positioned between the gingival tissue and the root surface to create and maintain a space for regeneration. Finally, the wound is closed to allow for periodontal regeneration.

The insertion of the subperiosteal implant generally involves one or more steps of the following: 1) making a lateral incision in the tissue covering the bony ridge (the incision can extend across the bone ridge), 2) tunneling the tissue such that it separates from the bone ridge both mesially and distally from the incision; the mesial and distal distance of the tunneled tissue are equal to the lengths of placed periodontal regeneration system, 3) injecting periodontal regeneration system under the tissue in the respective mesial and distal directions, 4) securing the two parts of the implant together by molding the implant to the bone under the tissue in the desired shape, 4) suturing the incision, and 5) subsequently inserting an subperiosteal implant system for later installing a post on the implanted prefabricated subperiosteal implant. One skilled in the art would be able to determine whether and/or which one or more of the foregoing steps are necessary for a particular periodontal regeneration.

Alternatively, a therapeutically effective amount of a growth factor can be applied along with the periodontal regeneration system. The growth factor can be, but is not limited to, one or more of the following: platelet-derived growth factor in a form having two beta chain (PDGF-BB), platelet-derived growth factor in a form having an alpha and a beta chain (PDGF-AB), IGF-I; and TGF-beta or their precursors in the form of either DNA or mRNA.

The periodontal disease can be any wound of periodontal disease which needs bone or tissue repair or regeneration. For example, the wound can be damaged bone, periodontium, connective tissue, or ligament of a mammal. In one embodiment, the defect is one of Class III furcation lesions or other periodontal tissue defects which result from periodontal disease, or other destructive or traumatic process to the periodontal tissue.

The methods of using the compositions disclosed herein will be further understood by reference to the following non-limiting examples.

### EXAMPLES

### Example 1

### Nano-HA or Micro-HA Particulate Augmented PPF Bone Graft

The bioactivity of a nano-hydroxyapatite-augmented, bioresorbable bone graft substitute made from the unsaturated polyester, poly(propylene fumarate) was analyzed by evaluating biocompatibility and osteointegration of implants placed into a rat tibial defect. Three groups of eight animals each were evaluated by grouting bone graft substitutes into 3-mm holes that were made into the anteromedial tibial metaphysis of rats. Two different formulations varying as to the type of hydroxyapatite were used: Group 1: nano-hydroxyapatite, Group 2: micron-hydroxyapatite, Group 3: control with HA only. Animals of each of the three groups were sacrificed in groups of eight at postoperative week three. Histologic analysis revealed superior biocompatibility and osteointegration of bone graft substitutes when nanohydroxyapatite was employed. At three weeks, there was more reactive new bone formation in this group when compared to the micron-hydroxyapatite group. The control group showed incomplete closure of the defect. This study demonstrated that nano-hydroxyapatite improves upon bioactivity of bone implant and repair materials. The model scaffold used in this study, poly(propylene fumarate), provided an osteoconduction pathway by which bone will grow in faster.

Materials & Methods

Materials and Formulations

The general formulation used for the study is shown in Table 1. PPF (Mw approximately 5,000 by GPC) was synthesized from equimolar fumaric acid and propylene glycol in the presence of p-toluene sulfonic acid, according to the methods of Gresser et al., J. Biomed. Mater. Res. 1995, 29: 1241-1247 and Gresser et al. Bone cement Part 1: Biopolymer for avulsive maxillofacial repair. In: Human Biomaterials Applications. Editors. Wise D L, Gresser J D, Trantolo D J, Yaszemski M J, Humana Press, Inc., Totowa, N.J., 1996, 169-187 (1996). 1-Vinyl-2-pyrollidinone (VP), Benzoyl peroxide (BP), hydroquinone (HQ), and N—N-dimethyl-p-toluidine (DMPT) were purchased from Aldrich (USA) and used as received. Sodium bicarbonate (SB), and citric acid (CA) were purchased from Fisher Scientific (USA)

The liquid component (part II) consisting of VP, accelerator DMPT, and distilled water was added to the dry powdered mixture (part 1) consisting of PPF, HA, SB, BP initiator, and CA to form a viscous putty-like paste resulting in a crosslinked polymer. The accelerator, DMPT, at a concentration of 0.03% w/w, gave a working time of about 90 seconds. The reaction of CA/SB with water produces carbon dioxide, the blowing agent responsible for pore formation and expansion. The stoichiometry requires a 1:3 mole ratio of CA:SB with a CA:SB weight ratio of 1.00:1.31, according to the method of Bondre, Tissue Engineering, 6(3): 217-227 (2000).

TABLE 1
General Composition of The Two-Part Formulation

| | |
|---|---|
| Part I (Wt., mg (Wt. %) | Part II (Wt., mg (Wt. %)) |

| | | | |
|---|---|---|---|
| PPF | 1179.5 (47.2) | VP | 380.0 (15.2) |
| HA | 341.5 (13.7) | DMPT | 0.65 (0.03) |
| SB | 51.3 (2.1) | H2O | 450.0 (18.0) |
| CtA | 43.4 (1.7) | Total | 845.65 (33.2) |
| BP | 52.5 (2.1) | | |
| Total | 1668.2 (66.8) | | |

PPF: Poly propylene fumarate
HA: Hydroxyapatite
BP: Benzoyl peroxide
SB: sodium bicarbonate
CtA: citric acid
VP: Vinyl pyrollidinone
DMPT: Dimethyl para toulidene
H2O: Distilled water

PPF foam with pore sizes of 100-300 microns appeared desirable for bone cell ingrowth. The reaction of CA/SB with water produces carbon dioxide, the blowing agent responsible for foam formation and expansion. The stoichiometry requires a 1:3 mole ratio of CA:SB with a CA:SB weight ratio of 1.00:1.31. The moles of CO₂, which can be generated per gram of material, depend on the loading of CA/SB in the foaming cement. A 0.15% CA/SB loading would produce a 25% expansion at 37° C. and 1 atm based on the above stoichiometry. In addition to the blowing agent, PPF formulation was crosslinked using vinyl pyrollidinone in the presence of an osteoconductive HA filler using techniques described by Bondre (2000).

The hydroxyapatites used in this study were: nano HA (group 1, median particle size=40 nanometers, as produced and characterized by Professor Ying at MIT) and 10 μm HA sintered, spherical micron-HA (group 2, median particle size=26 microns, commercially available from CAM Implants, The Netherlands) [Panchula and Ying in Nanostructured Materials: Science and Technology, edited by G M Chow and N I Noskova, (Kluwer, Netherlands, 1998), pp 319-333; Sun and Ying Nature 389: 704-706 (1997); Ying. Designer materials through nano processing in Frontiers of Engineering, (National Academy Press, Washington, D.C. 1996), 23-27; Ying, et al., J. Am. Ceram. Soc. 76(10): 2561-2570 (1993); Ying, et al., Angew. Chem. Int. Ed. 38(1): 56-77 (1999); Zhang, et al., Chem. Mater. 11(7):1659-1665 (1999); Zhang, et al., Chem. Commun. 1103-1104 (1999)]. The nano HA was compared to the micron HA and empty defects (Group 3), which were left to spontaneously heal. All HA preparations used in this study have been characterized using X-ray diffraction (XRD) to investigate the crystalline purity and size, photoacoustic Fourier transform infrared (PA-FTIR) spectroscopy to substantiate the molecular structure, and transmission electron microscopy (TEM) to determine the particle size and porosity.

In Vivo Animal Studies and Group Design

Three groups were tested in the rat tibial metaphysis implantation model according to Gerhart et al. J. Orthop. Res. 3,11: 250-255 (1993) using either type of HA available (Groups 1 and 2) and the unfilled control (Group 3). NIH guidelines for the care and use of laboratory animals (NIH Publication #85-23 Rev. 1985) were observed. Adult male Sprague Dawley rats weighing approximately 200 g were used as the animal model (Zivic Miller, Zelienople, Pa., USA). Animals were anesthetized using an intramuscular injection of ketamine HCl (100 mg/kg) and xylazine (5 mg/kg). The rats were also given an intramuscular prophylactic dose of penicillin G (25,000 U/kg), and the surgical site was shaved and prepared with a solution of Betadine (povidone-iodine) and alcohol (Dura-Prep; 3M Health Care, St. Paul, Md., USA). A 1.5 cm longitudinal incision was made in the anterior left hind leg, and the tibial metaphysis exposed. A 3-mm hole was made in the anteromedial tibial metaphysis of rats. The formulations, mixed prior to surgery to the consistency similar to a paste or putty, was implanted into the prepared tibial defect site with use of spatula. The PPF-based grout cured in situ and after the implantation of the bone grout, the soft tissues and skin were closed in layers with running absorbable sutures. A single formulation was implanted in eight animals. All the animals were sacrificed after three weeks postoperatively.

Methods of Evaluation

Evaluation was done by high-resolution radiographs taken immediately postoperatively and at three-week intervals until sacrifice using a specimen x-ray unit (Microfocus 50E6310F/G; Xerox, Rochester, N.Y., USA). Radiographs were taken with minimal exposure (32 kvp, 2 sec), and mammography film (Cronex Microvision; Dupont Medical Products, Wilmington, Del., USA), cassettes (MR Detail; AGFA Richfield Park, N.J., USA) and screens (Mammoray; AGFA) were employed. Following sacrifice, 10-mm-long segments of the tibial bone including the section that was implanted with a bone graft substitute were harvested. The specimens were processed for histologic analysis by fixation in 10% buffered formalin. Specimens, which included residual bone graft material, were decalcified in EDTA and paraffin embedded. Longitudinal sections (5 μm thick) of the total specimen were then cut and stained with hematoxyline and eosin. In addition, slides were stained with the von Kossa method to demonstrate calcium crystals. Slides were examined for resorptive activity and new bone formation at the implantation site, as well as for inflammatory responses.

Histomorphometric evaluation of new bone formation around the different types of grafts was done by acquiring images of serial longitudinal hematoxyline and eosin stained sections of the specimen using a CCD video camera system (TM-745; PULNiX, Sunnyvale, Calif., U.S.A.) that was mounted on a Zeiss microscope. Images were digitized and analyzed using Image Pro Plus software. For each specimen, the area of newly formed bone surrounding the implant and within the implant was measured. This measurement was standardized against the total area occupied by the implant in the same section. A minimum of five sections obtained from different levels of the specimen was included for this analysis. The spacing between sections of adjacent levels was typically 300 micrometers, allowing an approximate absolute volume of the newly formed bone, which is given as an average percentage rate (mean±standard deviation) of these volume measures for each bone specimen to be obtained. To compare the extent of new bone formation around the implant at its metaphyseal site between the experimental groups and the control group, the recovery index was determined. It was defined as the volume ratio of newly formed bone and the volume of the whole implant based on eight animals per study group. They are thus given as average percentage rates.

Statistical Analysis

Differences in the remodeling indices were analyzed for statistical significance by employing an ANOVA test. A p-level of 0.05 was considered statistically significant.

Results

Control Group:

In the control group, new bone formation in the metaphyseal defect made in the rat tibia was absent. There was some periosteal bone formation at the cortical drill hole site, but the remainder of the defect in the tibial metaphysis was filled primarily with bone marrow and fatty tissue.

In the micron-hydroxyapatite group, the implant remained structurally stable and did not disintegrate. There was no histologic evidence for implant dissolution or active cellular resorption from the recipient site. Although there was some moderate infiltration with PMNs, this seemed consistent with postoperative inflammatory changes. In addition, there was new bone formation, which at three weeks postoperatively appeared tightly packed around the implant without excessive fibrous or inflammatory tissue. There was osteoclastic and osteoblastic activity at the surface of the implant suggesting that the bone surrounding it was undergoing active remodeling. Although the bone surrounding the implant bone graft underwent active remodeling, the implant remained structurally intact. Micron-hydroxyapatite crystals were easily demonstrated with the von Kossa stain. New bone formation and large round cells whose morphologic appearance was consistent with osteoblasts were noted in close proximity to the micron-HA crystal.

In the nano-hydroxyapatite group, the implant surface stimulated a more vigorous inflammatory response with infiltration by PMNs and macrophages. In addition, there appeared to be more new bone formation around the implants. Similar to Group 2, in Group 3 there was also no histologic evidence for implant dissolution or active cellular resorption from the recipient site. In contrast to the micron-hydroxyapatite group, no HA crystals were stainable with the von Kossa technique in the nano-hydroxyapatite group. Similarly, as in the micron-HA group, large cells with a round nucleus, positioned towards the interface with the implant, were present. Osteoids appeared to be secreted on the implant material.

Histomorphometry showed that the amount of new bone formed around the different types of grafts used in this study was significantly higher in the nano-hydroxyapatite group than in the control group (no implant; p<0.002) and in the micron-HA group (p<0.025). Although both formulations were equally osteoconductive, as measured by the implant area covered by newly formed woven bone, a wider margin of newly formed bone was noted around nano-hydroxyapatite implants. In addition, more new bone was found within these types of implants. As a result, the remodeling index was higher in the nano-hydroxyapatite group when compared to the micron-hydroxyapatite group (see Table 2).

TABLE 2
Histomorphometric Analysis of New Bone Formation

| Groups | Recovery Index [%] |
|---|---|
| Empty defect | 12.3 ± 6.9 |
| Micron-HA-PPF implant | 34.3 ± 10.6 |
| Nano-HA-PPF implant | 48.3 ± 14.9 |

The ultimate objective of this animal study in rats was to establish the utility of nano-hydroxyapatite use in an osteoconductive grout while demonstrating biocompatibility in the absence of foreign body reactions and evaluating the effect on the bone healing/repair process. The PPF-based resorbable bone graft substitute presented here was expected to be osteoconductive because the hydroxyapatite filler has been successfully employed in similar model evaluations.

Two types of HA particle sizes were investigated in this study: (a) sintered, spherical nano-HA (median particle size=40 nanometers), and (b) sintered, spherical micron-HA (median particle size=26 microns). Results of this study showed no new bone formation in negative controls, which were not filled with any graft material. At three weeks, there was more reactive new bone formation without complete closure of the defect in the nano-HA group than in the micron-HA group. These histologic observations were supported by histomorphometric measurements of new bone formation that demonstrated significant increases when PPF was used in combination with nano-HA (Table 2). For the time period analyzed in this rodent study (three weeks), there was no evidence of implant failure or disintegration. In addition, it was clearly evident that these PPF-based bone graft substitutes were extremely osteoconductive showing both ingrowth of newly formed woven bone with concurrent neovacularization.

On the basis of these observations, this study showed that the osteoconductive properties of a PPF-based bone graft material containing 13.7% HA can be further improved by utilization of nano-hydroxapatite. Rapid bony ingrowth and healing could be facilitated by accelerated bone formation around and within a biodegradable scaffold.

### Example 2

### Repair of Periodontal Defect with PPF Containing HA Particles

Materials and Methods

Poly(propylene fumarate) was synthesized by the direct esterification of fumaric acid (Fisher Scientific, Inc.) with propylene glycol (Aldrich Chemical Co., Milwaukee, Wis.) as described above. Briefly, the reaction was catalyzed by p-toluene sulfonic acid monohydrate (Aldrich Chemical Co., Milwaukee, Wis.) in the presence of t-butyl hydroquinone (Aldrich Chemical Co., Milwaukee, Wis.), an inhibitor of spontaneous crosslinking at elevated temperatures. The reaction product was dissolved in methylene chloride, filtered to remove unreacted fumaric acid, washed with 20% aqueous methanol to remove unreacted propylene glycol and dried over type 3A molecular sieves (EM Science Co.). The polymer was recovered from the methylene chloride by precipitation into di-ethyl ether, redissolved in acetone, dried and filtered and the acetone removed under vacuum. The weight-average molecular weight of PPF was determined by gel permeation chromatogrphy, using a 7.8×300 mm ultrastyragel 10³ angstrom column (Waters, Model 410, Milford, Mass.), to be 6650 with a dispersity of 2.57 [17,20]. Hydroxyapatite (ha, CAM Implants BV, The Netherlands) was made in micro or nano form. N-vinyl pyrrolidone (VP) (Aldrich Chemical Co., Milwaukee, Wis.) was vacuum distilled (93° C., 13 MM Hg) to remove the NaOH inhibitor. The VP in solution with n,n-dimethyl-para-toluidine (DMPT) (Aldrich Chemical Co., Milwaukee, Wis.) and Tween 80 (Fisher Scientific, Inc.) was added to a dry powder mixture of PPF and HA to form a viscous tan colored putty. Sodium bicarbonate (Fisher Scientific, Inc.), benzoyl peroxide initiator (Aldrich Chemical Co., Milwaukee, Wis.), and citric acid (6.8 wt % solution) (Fisher Scientific, Inc.) were added in turn to create an osteoconductive foaming network.

The unsaturated PPF polymer can be crosslinked with VP in the presence of effervescent agents, sodium bicarbonate (SB) and citric acid (CA), and HA to create an osteoconductive foaming network, which can be mixed with a bone graft immediately prior to defect filling. The reaction of citric acid and sodium bicarbonate with water produces carbon dioxide, the blowing agent responsible for foam formation and expansion. A SB/CA loading of 1% will generate an expansion of around 200% at 37° C. and 1 atm based on stoichiometric release of CO₂ according to the following reaction:

HOC(CO₂H)(CH₂CO₂H)+3NaHCO₃→3H₂O+3CO₂+HOC(CO₂Na)(CH₂CO₂Na)₂

Using a testing machine equipped with a 500 lb. load cell and operating at a cross head speed of 1 cm/min, the mean strength and modulus were 17.7±2.8 MPa and 365.3±74.9 MPa, respectively. These compressive strength data are comparable with values reported by Carter and Hayes, who measured this property of bone as a function of strain rate and density. At a comparable strain rate, the compressive strength of trabecular bone (ρ=0.31 g/cm³) was noted to be 5.0 MPa, and for cortical bone (ρ=2.0 g/cm³) was 200 MPa.

Degradation Mechanics

Polymer degradation occurs by hydrolysis of the ester linkages resulting in chain scission (Gresser et al., 1995; Peter et al., 1997a, 1997b). The quantitative development of in vitro mechanical data focuses on the desired in vitro outcomes now defined as initial compressive values comparable to cancellous bone (5 Mpa for the strength and 50 Mpa for the modulus) with a rate of mechanical loss not to exceed 50% of initial values at three weeks.

In vitro assessment of biomechanical properties of scaffolds in relation to polymer degradation was carried out to correlate the temporal sequence of polymer degradation with the mechanical strength of the graft material to determine the influence of the polymer degradation to mechanical loss over time. Samples for modulus and strength testing were incubated at 37° C. in phosphate buffered saline (as described in ASTM Method F1634-95, “Standard Practice for In-Vitro Environmental Conditioning of Polymer Matrix Composite Materials and Implant Devices”) under load to approximate physiological conditions and removed at 4 days and at 1, 2, 3, 6, 9 and 12 weeks. Protocols for sample loading in vitro were based on an adaptation of ASTM Methods F451-99a (specification for acrylic bone cements) and D5024-95a and D4065-95 (for measuring and reporting dynamic mechanical properties) as suggested in the FDA Guidance Document for Testing Implant Devices (April 1996) and previously used for the in vitro testing of biopolymeric fixtures under load (Trantolo et al., 2000). Upon removal from the bath, samples were maintained in a fully hydrated condition in saline-soaked gauze. Standard procedures employed by Dr. Wilson C. Hayes in the Oregon Health Sciences University laboratories were used to determine the mechanical values of the PPF foam extenders in the in vitro evaluations. Automated data acquisition was employed throughout the study using Lab View™. All experiments were conducted under Good Laboratory Practice (GLP) guidelines, with Standard Operating Procedures (SOP's) for each protocol and data analysis as required by FDA guidelines.

An analysis of variance was used to detect statistically significant differences in the rate of mechanical loss measured at each study interval. A paired t test was used to compare micron-HA formulations with nano-HA formulations. In all statistical tests employed, a significance level of p<0.05 was chosen.

Formulations selected on the foregoing mechanical basis were subject to morphological characterization via scanning election microscopy (SEM, AMR-1000, Advanced Metals Research Corp.). A temporal profile was developed using six samples of each selection at each of four time points (t=0, 1, 3, and 6 weeks) with data processed and analyzed using NIH Scion Image software.

Evaluation Procedures

The PPF-based grouts that were either augmented with nano-HA or with micro-HA were screened and compared to controls. Rats were divided into four groups, where each of three groups were implanted with either a micron-HA PPF implant (group 1), a nano-HA PPF implant selected on the mechanical outcome of Task 2 (group 2), or demineralized bone (group 3). A fourth group (Group 4) was a sham control (i.e., an unfilled defect) to evaluate spontaneous healing of the defect model. Forty-five-day old Sprague-Dawley rats (Zivic Miller, Zelienople, Pa., USA.) were randomly divided into 4 groups of 36 animals each.

For tooth extraction, animals were weighed and then anesthetized with a single intraperitoneal injection of sodium pentobarbital (Nembutal) 50 mg/kg body weight. A sharp explorer was used for extractions. All maxillary and mandibular molars were extracted on the right side under a dissecting microscope (Dentiscope, Johnson & Johnson, E. Windsor, N.J.). The periodontal ligament was then loosened from the cervical portion of each tooth by running the tip of the explorer around each tooth and gently separating the tissue. The explorer was then placed interproximally between the molars and luxated out.

In animals in the experimental groups, the mandibular site was packed with the different PPF-based implant materials or the demineralized human bone matrix commercially available as Grafton Putty® from the Musculoskeletal Transplant Foundation. Sockets were gently packed with a small amalgam condenser after placement of the material into the sockets with an amalgam carrier. Interrupted sutures were placed to close the extraction sites and to keep the implant material in place. The animals were fed soft rat chow and water ad libitum.

Twelve animals from each group were sacrificed at 3, 6, and 16 week postoperatively. Animals were injected with a fluorescent dye two weeks prior to sacrifice for histolgoic evaluation as described below. A total of 144 rats were included.

Evaluation were performed by using radiographic, histolgoic and histomorphometric techniques, which are described below:

Radiographs:

Standardized radiographs (0.3 sec, 80 kVp) were made a 1 day, and at 2, 4, 8, and 16 weeks after extraction. Two radiographs were made on both the right and left sides of the mandible. An acrylic cone guide was fixed to the cephalostat to standardize the angle of the x-ray beam. The radiographs were scanned and digitally analyzed with Image Pro Plus Software to measure the residual ridge height as described by Nishimura et al. (1987). As earlier studies have demonstrated, films made by this technique are virtually superimposable.

Histologic Examination:

Animals were sacrificed at week 3, 6, and 16 weeks after extraction and PPF-implantation. The mandibles were immediately removed, skinned, and fixed in 10% formalin for more than 24 hours. Specimens were trimmed to include only the molar ridge region and were then placed in ethylenediaminetetraacetic acid (EDTA) until they were fully decalcified. Specimens were sectioned along the length of the ridge in a mesiodistal direction and embedded in paraffin. Slices 5 μm thick were made and stained with hematoxylin and eosin.

Histomorphometry:

Histomorphometry was then performed on all specimens. For this purpose, a CCD video camera system (TM-745; PULNiX, Sunnyvale, Calif., USA) mounted on a Zeiss microscope was used. Images were digitized and analyzed using image software (ImagePro Plus). For each calvarial tissue specimen, the area of the defect (i.e. implant) substituted by newly-formed bone divided by the total area of the defect were determined on sequential histologic sections. The spacing between sections of adjacent levels were typically 300 micrometer. A minimum of ten sections were included for this analysis. Data were presented as an average percentage rate (mean standard deviation).

Statistical Analysis:

The Mann-Whitney test was performed to determine statistical analysis of the change of the residual ridge height between untreated control and implant treated groups. A Mann-Whitney test was also performed on the net weight gain after extraction in the untreated control group and the implant-treated group testing the null hypothesis that net weight gain would be the same for both groups. The resorption pattern for each animal was determined to fit into a resorption curve fit to a second order equation y=ax²+c. Multiple linear regression analysis was performed on each animal used in the study. The coefficient of determination (R²) shows suitable fit. R² is a proportion and takes on values in the range of 0 to 1; the closer the value of R² to 1, the better the fit of individual data to a resorption model. The third analysis is a study of the changes in outcome at specific times by using Kruskal-Wallis analysis. This nonparametric one-way analysis of variance was applied at specific times during the study (3, 6 and 16 weeks after extraction) to the untreated control group and the implant-treated group to determine whether the rate of resorption between groups differed. Lastly, a t-test was used to measure the change in ridge height between week 3 and week 16 after extraction. The test was applied separately to the untreated control group and the implant treated group.

The formulations were evaluated by implanting grafts into the mandibular site of rats.

Results

A porous scaffold was formed by crosslinking of the unsaturated PPF polymer with a vinyl monomer, vinyl pyrrolidone (VP), in the presence of the effervescent fillers, sodium bicarbonate and citric acid, and the osteoconductive filler, HA. Upon mixing, the mixture cured via crosslinking of the PPF by the monomer and concomitant CO₂ generation resulting in a porous scaffold degradable by hydrolysis. The use of HA as part of the filler supported the osteoconductivity of the scaffold (Saito, Biomaterials 15, 156-160 (1994), while the CO₂ generated pores provide porous regions for attachment and proliferation of cells in situ (Bondre et al., 2000) and the hydrophilicity of the polymeric support encourages cellular migration (Lewandrowski et al. 1999).

This PPF scaffold (without HA particulate) had been evaluated in vitro for morphological, mechanical and surface properties and in vivo using a rat tibial defect model (Gerhart et al., 1989). Scaffolds, designed for controlled superstructural characteristics, were shown to be hydrophilic, mechanically comparable to trabecular bone (6.4 MPa for the compressive strength of the PPF graft material versus 5.0 MPa for that of trabecular bone (Carter and Hayes, Science 194, 1174-1175 (1976)), dimensionally stable, and porous (Bondre et al., 1999). Histologic and histomorphologic examination of the implant region of rats showed that the porosity of the scaffold supported bony ingrowth and the stability of the scaffold preserved the dimensional integrity of the defect site (Lewandrowski et al, 1999). Preliminary mechanical tests on a nano-HA loaded PPF filler showed that a PPF composite loaded with nano-HA has a compressive strength of 7.5 MPa.

### Example 3

### PLA-Based Scaffolds with Micro or Nano HA for Spinal Segment Repair

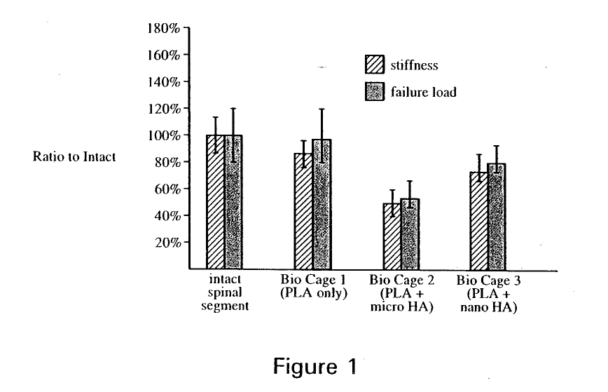

Poly(lactic acid), PLA, fixtures intended for spinal fusion were filled with HA, either micron-sized or nano-sized, and compared to PLA-only fixtures. FIG. 1 shows the effect of HA-filling on the compressive properties of this fixture in spinal segments after discemtomy L4/L5 and dissection of the anterior and posterior longitudinal ligaments. FIG. 1 summarizes failure load and stiffness results normalized with respect to the intact motion segment during axial compression. The polymer-only figure (“Bio cate 1”) was 80-10% lower in stiffness and failure load, respectively, compared to an intact spinal segment, while the micron-HA filled “Bio cage 2” was 45-50% of those values. The “Bio cage 3,” loaded with nano-HA, was statistically equivalent to the unfilled polymer (“Bio cage 1”).

### Example 4

### PPF-HA for Use as Bone Graft Extender

A scaffold formulation based on PPF was used as bone graft extender in a rat tibial model. The scaffold was mixed with autograft and allograft material and placed directly into a cylindrical metaphyseal defect made into anterior aspect of the rat tibia using a dental cutter (measuring 4.5 mm in diameter). The material was allowed to cure in situ. These formulation were compared to defects without any graft material, autografts, allografts and PPF alone.

Materials and Methods

Six groups of animals comprised the study. Fresh corticocancellous autografts were procured intraoperatively and then immediately mixed with the PPF-bone graft extender (see Table 3) prior to reimplantation to the defect site. Allografts of similar nature were procured from Sprague-Dawley rats, cleaned of soft tissues and bone marrow, fresh frozen and kept at −80° C. until use. Both auto- and allografts were mixed with the PPF-bone graft extender at a ratio of 50:50.

TABLE 3
Component of bone graft extender formulation

| Chemical | Wt. % |
|---|---|
| PPF | 46.10 |
| Hydroxyapatite | 13.40 |
| Vinyl pyrrolidone | 12.10 |
| DMPT | 0.31 |
| Tween 80 | 0.41 |
| Sodium bicarbonate | 2.24 |
| Benzoyl peroxide | 0.84 |
| Citric acid (6.8 wt %) | 24.60 |

Male Sprague-Dawley rats (approx. 400 grams, Charles River Breeding Laboratories) were used as the animal model. Animals were anesthetized using an intramuscular injection of ketamine HCl (100 mg/kg) and xylazine (5 mg/kg). The rats were also given an intramuscular prophylactic dose of penicillin G (25,000 U/kg), and the surgical site was shaved and prepared with a solution of Betadine (povidone-iodine) and alcohol (Dura-Prep; 3M Health Care, St. Paul, Md., USA). Sets of 3 and 6 animals were sacrificed at 1 and 4 weeks, respectively, for each of the 6 groups investigated. Thus, a total of 54 animals were included in this study.

Results

Histologic evaluation of negative controls, which were not filled with any graft material, showed filling of the defect with fibrous and granulation tissue at one week postoperatively. At four weeks, there was some reactive new bone formation without complete closure of the defect. Defects filled with either PPF or autograft bone material alone were used as positive controls. These sections showed early new woven bone formation in the autograft-group with near complete healing of the defect at four weeks. Defects filled with PPF alone showed early new bone formation being promoted by the PPF foaming scaffold demonstrating the osteoconductive properties of the material. Increasing resorption of the material with subsequent gradual ingrowth of new bone was seen at four weeks postoperatively.

Mixing of the PPF bone graft extender with either allograft or autograft material resulted in enhancement of new bone formation with both all- and autograft. However, improved osteoinduction was only seen when the PPF bone graft extender was mixed with fresh autograft. The combination of PPF with autograft resulted in more new bone formation than when autograft was used alone. In addition, there was more new bone when a combination of allograft versus allograft alone was used. These histologic observations were supported by histomorphometric measurements of new bone formation which demonstrated significant increases when PPF was used in combination with either auto- or allograft (Tables 4 and 5). The metaphyseal and cortical remodeling indices were determined as approximated average percentage rates based on 3 or 6 animals (for the 1 week or 4 week time points respectively) per study group. This analysis showed that there was significantly more new bone formation in the experimental groups (bone graft mixed with PPF bone graft extender) when compared to the positive control groups (bone graft or PPF bone graft extender alone).

TABLE 4
Histomorphometric analysis of new bone formation at the
metaphyseal drill hole defect

| | Metaphyseal remodeling index (%) | |
|---|---|---|
| Groups | 1 week postoperatively | 4 weeks postoperatively |
| Empty defect | 8.5 ± 4.5 | 12.3 ± 6.9 |
| PPF alone | 32.7 ± 7.9 | 42.1 ± 11.2 |
| Autograft alone | 57.9 ± 17.9 | 67.1 ± 19.3 |
| Allograft alone | 37.9 ± 14.6 | 45.1 ± 21.4 |
| PPF + autograft | 46.7 ± 15.9 | 83.1 ± 19.9 |
| PPF + allograft | 41.2 ± 12.9 | 52.1 ± 16.1 |

TABLE 5
Histomorphometric analysis of new bone formation at the
metaphyseal drill hole defect

| | Metaphyseal remodeling index (%) | |
|---|---|---|
| Groups | 1 week postoperatively | 4 weeks postoperatively |
| Empty defect | 4.5 ± 4.5 | 8.3 ± 6.9 |
| PPF alone | 22.7 ± 6.5 | 32.7 ± 9.2 |
| Autograft alone | 39.2 ± 14.5 | 52.6 ± 18.5 |
| Allograft alone | 33.9 ± 13.2 | 46.1 ± 16.6 |
| PPF + autograft | 51.2 ± 18.2 | 79.4 ± 21.8 |
| PPF + allograft | 49.2 ± 16.3 | 48.2 ± 15.7 |

### Example 5

### Use of PPF-HA Foam for Periodontal Tissue Regeneration as Immediate Implant Support

Materials and Methods

A bone graft extender carrier was prepared as a foaming putty which is prepared as a two part system in order to separate the polymerizable entities (PPF, VP) from the initiator (BP). Because BP is, by weight, a minor ingredient, it is packaged with components inactive with respect to benzoyl peroxide to provide bulk and with the inhibitor.

The foaming agent consists of granules composed of a stoichiometric ratio of citric acid (CA) and sodium bicarbonate (i.e., 1.0:1.3 w/w). The in vitro test specimens were circular cylinders prepared by placing the polymerizing composite material into a 10 mm diameter, 10 cm high cylindrical Teflon™ mold and letting the polymer foam crosslink. Samples used for the mechanical testing were 0.0 mm×10 mm for the in vitro and in vivo testing. The example formulation yielded a foam with a density of 0.6484±0.0834 g/ml.

Mechanical testing was done in accordance to ASTM F451-95 for bone cements. The samples were tested in compression on an Instron Model 8511 Materials Testing Machine. The Instron was fitted with a 500 lb load cell, and the load measuring apparatus was zeroed, balanced and calibrated. The samples were compressed to failure at a crosshead speed of 1 cm/min and the load deformation curve was recorded. From these data the ultimate compressive stress (σ) and Young's modulus (γ) could be calculated. The ultimate compressive stress was calculated as the applied load at failure divided by the original cross-sectional area of the rest specimen whereas the Young's modulus was calculated as the slope of the load deformation curve in its linear portion. The mean compressive strength (mean of 8 samples) of the porous cement was 6.77±2.5 MPa. These compressive strength data are comparable with values reported by Carter and Hayes, who measured this property of bone as a function of strain rate and density. At a comparable strain rate, the compressive strength of trabecular bone (ρ=0.31 g/cm³) was noted to be 5.0 MPa (carter and Hayes, 1976).

In order to insert this implant system, only a simple surgical session is required necessitating a bucco-lingual incision made in the gum tissue at the center of the edentulous span. Then a tunneling procedure was used to separate the tissue from the alveolar ridge crest in both directions from the incision. This was done without an additional incision through the tissue and without reflecting the tissue. After the tissue has been separated from the bone, the polymer system was injected completely underneath the tissue distal to the incision. Then, the expanding system was being molded forward or anterior to the incision underneath the tissues until the desired shape of the periodontal tissues to be augmented or “erected” was achieved. This shaping under the gum tissues hardened the biodegradable polymer system so that it assumed a relatively rigid structure.

Once the periodontal regenerate system had been formed over the alveolar ridge crest of the bone, and was in close conformity therewith, the incision were sutured so that the implanted regenerate system is completely out of operation. During this procedure, a subperiosteal implant system was placed. A period of time was then allowed to pass, during which the gum tissue re-established itself. Then, the post and an artificial tooth structure was installed on top of the subperiosteally placed implant system. The implant system was tunneled to both sides of the incision, one half was slipped under the tissue in one direction, and the other half was slipped under the tissue in the other direction. The two injected implant systems were then brought together and molded to one piece.

Once sufficient time has passed for the implant portions to be firmly held in place, a coping for an artificial tooth structure was fabricated. This coping was screwed over the outer threads which were provided by the subperiosteally placed implant and acted as the posterior crown of a fixed prosthesis.

Results

A dimensionally stable porous scaffold was prepared by crosslinking the unsaturated PPF polymer with a vinyl pyrrolidone (VP) in the presence of sodium bicarbonate and citric acid and hydroxy apatite (HA). To demonstrate feasibility, the PPF scaffold was evaluated in vitro for morphological, mechanical and surface properties, and in vivo using a rat tibial defect model established by Gerhart et al. (1989), as described above. Scaffolds were shown to be mechanically comparable to trabecular bone, dimensionally stable, and porous. Histologic and histomorphologic examination of the implant region of rats suggested that the scaffold of the biodegradable bone graft extender supported bony ingrowth and the stability of the scaffold preserved the dimensional integrity of the defect site.

The temporal sequence of bone ingrowth into PPF-foaming scaffold injected into tibial drill holes was investigated. Osteoclastic and osteoblastic activity and neovascularization was seen at the foam implantation site as early as 1 week postoperatively. It appeared that the foam served as a scaffold for new bone growth. At postoperative week three, the drill hole was completely healed in all animals injected with the foam. In comparison, this was not the case in control animals in which only a hole was drilled but no implant was injected. Although there was some periosteal bone formation at 3 and at 4 weeks postoperatively, complete healing of the hole was not observed in any of the sham operated animals.

### Example 6

### Determination of Osteoconductive Properties of a Porous Poly(propylene glyco-glycol-co-fumaric Acid) Scaffold

Materials & Methods

Materials

Poly(propylene fumarate) (PPF) was synthesized by the direct esterification of fumaric acid (Fisher Scientific, Inc., Pittsburgh, Pa., USA) and propylene glycol (Aldrich Chemical Co., Milwaukee, Wis., USA) in the presence of p-toluene sulfonic acid (Aldrich) (Gresser, et al. J. Biomed. Mat. Res., 29, 1241-1247 (1995); Lewandrowski, et al. Tissue Engineering Tissue Eng;5(4):305-16 (1999)). 1-vinyl-2-pyrollidinone (VP), benzoyl peroxide (BP), and N-N-dimethyl-p-toluidine (DMPT) were purchased from Aldrich and used as received.

Bone Repair Material Formulation

The PPF-based bone graft substitute system was prepared as a two-part formulation consisting of solid powder and liquid components as shown in Table 6. The bone repair system was prepared by mixing an aqueous solution of VP (72.6% w/w) and DMPT (0.2% w/w) to a dry powdered mixture of PPF (71.8% w/w) and hydroxylapatite to form a viscous putty-like paste. The weight ratio of PPF:VP was kept constant at 4:1. The crosslinking reaction between PPF and VP was initiated by the addition of benzoyl peroxide (BP; 3.6% w/w). Generation of free radicals was accelerated through the use of DMPT in the liquid mixture. Sodium bicarbonate (1.7% w/w) and citric acid (1.3% w/w) were also added to the dry powder formulation. Upon mixing of the VP solution and PPF powder, the reaction of the effervescent agents citric acid (CA) and sodium bicarbonate (SB) resulted in controlled expansion of the graft material with respective pore sizes of 100 to 1000 μm.

Two types of hydroxylapatite (HA) were used to create two specific PPF-formulations: sintered, spherical μm-sized HA (median particle size=26 μm, commercially available from CAM Implants, The Netherlands) and nm-sized HA (median particle size=40 nm (Sun T and Ying J Y. Nature, 1997, 389: 704-706 (1997); Ying, et al. J. Am. Ceram. Soc. 76(10): 2561-2570 (1993); Ying et al. Angew. Chem. Int. Ed. 38(1): 56-77 (1999); Zhang, et al. Chem. Mater. 11(7): 1659-1665 (1999)). As outlined below, the μm-sized (Group A) and nm-sized HA (Group B) PPF formulations were compared to defects filled with demineralized bone matrix (Group C) and empty defects left to heal unaided (Group D).

TABLE 6
Composition of PPF-Based Bone Repair System

| | |
|---|---|
| Chemical | Amount [% w/w] |

Solid Components

| | |
|---|---|
| Poly(propylene fumarate), PPF | 71.8 |
| Hydroxylapatite, HA | 21.6 |
| Benzoyl peroxide, BP | 3.6 |
| Sodium bicarbonate, SB | 1.7 |
| Citric acid, CA | 1.3 |

Liquid Components

| | |
|---|---|
| 1-Vinyl-2 pyrollidone, VP | 46.0 |
| Water | 53.8 |
| N-N-dimethyl-p-toluidine, DMPT | 0.2 |

Design of Animal Studies

To evaluate the osteoconductive effect as well as the biocompatibility of the PPF-based bone repair material, formulations were implanted into noncritical calvarial defects using a rat calvarial defect model previously described by Pettis, et al. J. Oral Maxillofac Surg 48(10):1068-74 (1990) and Salata, et al. Int. J. Oral Maxillofac Implants 13: 44 51 (1998).

The size of the defects measured four mm in diameter. National Institutes of Health guidelines for the care and use of laboratory animals were observed. Sprague Dawley rats weighing approximately 100 g and 28 days of age were used as the animal model (Charles River Laboratories, Wilmington, Mass., USA). Animals were anesthetized using an intramuscular injection of ketamine HCl (100 mg/kg) and xylazine (5 mg/kg). The surgical site was shaved and prepared with a solution of Betadine (povidone-iodine) and alcohol (Dura-Prep, 3M Health Care, St. Paul, Minn., USA). The rats were also given an intramuscular prophylactic dose of penicillin G (25,000 U/kg) postoperative.

Two 4-mm diameter cortical defects were produced in each rat skull. Animals were divided into three groups of 8 animals and one defect was treated with one of the following: PPF formulation containing μm-sized HA (Group A), nm-sized HA and PPF (Group B), or demineralized bone matrix (Group C). The second defect was left to heal unaided and serve as a paired control. Animal groups were evaluated at 1, 2, 4, and 7 weeks postoperatively, hence, a total of 96 animals was included in this study. The PPF formulations were mixed during surgery to the consistency similar to a paste or putty, and then implanted into the prepared cranial defect site with use of spatula. The bone repair material formulation was cured in situ. Demineralized bone matrix (DMB, Grafton Putty™, Muskuloskeletal Transplant Foundation, Shrewsbury, N.J.) was obtained for implantation in Group C animals. Implant materials were allowed to cure in situ for approximately five min., and then the soft tissues and skin were closed in layers with running absorbable sutures.

Methods of Evaluation of the Bone Repair Material Formulations

Following sacrifice, excision biopsies of the skull and surrounding soft tissues were radiographed. Then the specimens were fixed in 10 percent neutral buffered formalin and decalcified in 4 N formic acid. Serial longitudinal sections 5 μm thick at 50 μm intervals were produced. Sections were stained with haematoxylin-eosin (H&E). Slides were examined for defect healing by descriptive analysis of the resorptive activity and new bone formation at the cranial defect site, as well as for inflammatory responses to the bone repair material. In addition, histomorphometric evaluation of new bone formation in response to the cranial defect and implantation of the PPF repair material was done by acquiring images of serial longitudinal sections of the specimen using a CCD video camera system (TM-745; PULNiX, Synnyvale, Calif., U.S.A.) mounted on a Zeiss microscope. Images were digitized and analyzed using Image Pro Plus software. The areas occupied by new bone in the defect were quantified using H&E-stained slides, from two animals at 1, 2, 4, and 7 weeks. The new bone formation, expressed as a percentage of the area of the original 4-mm defect compared to the empty control of each animal, was calculated for each sample using three templates, or region of interest masks, placed in three areas across the cranial defect and a corresponding contralateral area in the control samples. A mean was obtained for each sample from a minimum of three and a maximum of six serial longitudinal sections. This allowed obtaining an approximate absolute volume of the newly formed bone, defined as the New Bone Volume Index, which is given as an average (mean±standard deviation) of these consecutive area measures for each bone specimen. New Bone Volume Index is given as a percentage rate and is presented as the average of all sections prepared from the PPF-implanted animals per group.

Statistical Analysis

Differences in the amount of new bone formed in response to implantation of PPF formulation containing either μm-sized HA (Group A), or nm-sized HA (Group B) were analyzed for statistical significance by employing an ANOVA test for normally distributed samples. The Tukey test was used to establish P values. The Kruskal-Wallis test was used to analyze non-normally distributed samples. A p-level of less than 0.05 was considered statistically significant.

Results

In the 96 experimental implantation sites, there were no postoperative complications or clinical signs of implant reaction. No fractures or deep infections were observed over the entire postoperative period. Specimens were inspected macroscopically after having been dissected, and prior to sectioning and embedding for histologic, and histomorphometric analysis.

Results are depicted in Tables 7 and 8.

TABLE 7
Subjective Scoring of Inflammatory and Multinucleated
Giant Cells at 4 Weeks

| Cells / Material | Score 4 weeks n = 10 |
|---|---|
| Inflammatory Cells | |
| PPF Implant | 2.12 ± 0.48 |
| Empty Control | 1.6 ± 0.34 |
| Giant Cells | |
| PPF Implant | 0.94 ± 0.27 |
| Empty Control | 0.12 ± 0.04 |

TABLE 8
New Bone Volume Index for each graft type based on 8 rats
per group and 4 weeks postoperative follow up.

| | New Bone Volume Index [%] |
|---|---|
| nm-HA/PPF | 95 ± 17 |
| μm-HA/PPF | 67 ± 12 |
| DBM | 46 ± 5 |

All implanted specimens were found to be filled with variable amounts of newly formed bone. No empty defect sites were found. All PPF- and DMB-grouted bone specimens were retrieved intact. The implantation of the PPF-based bone repair material material into cranial defects resulted in overall benign tissue responses as evidenced by the absence of excessive macroscopic granulation tissue formation in any of the retrieved specimens. All surgical sites appeared to have healed well and there was no apparent adverse reaction of the surrounding soft tissues to the in situ cured material. All of the control defect had benign appearing soft tissue reactions.

Radiographic Studies

Radiographic analysis of all experimental cranial specimens at the various follow-up time points showed that there was sufficient evidence of bone healing at the implantation sites by four weeks postoperative regardless of which implant materials was used. There were no radiographic lucencies in and around the defect sites. At four weeks, radiographs showed more bone formation in the two PPF-based groups than in the DMB group. There was no evidence of bone formation in the control defects that were left to heal unaided. The amount of new bone formation was semiquantitatively evaluated with light absorbance measurements which were standardized between radiographs with an internal phantom. The measurements showed the highest amount of new bone formation in the PPF implant containg nm-sized HA. On radiographs, the surrounding soft tissues were normal in appearance without any evidence of swelling or fluid collections at all implantation sites.

Histologic Analysis

In Group A, PPF-based bone repair material containing μm-sized HA had been implanted. Histologic analysis of the bone samples retrieved at 1 and 2 weeks postoperatively showed that the in situ cured bone repair material remained largely intact. There was some new bone formation, which occurred in a centripetal fashion from the periphery of the defect towards the center. At 4 and 7 weeks postoperatively, the PPF implant appeared to have been increasingly replaced by newly formed bone. The PPF implant was no longer intact and degradation followed by bony ingrowth appeared to have occurred. In most samples, defects were noted to heal but were not completely filled with new bone.

In Group B, PPF bone repair material containing nm-sized HA had been implanted. Histologic findings at 1 and 2 weeks postoperatively were similar to Group A samples. However, bone formation appeared more vigorous with a more pronounced initial inflammatory response as evidenced by the accompanying bone marrow proliferation within the newly formed bone trabeculae surrounding the implant. At four weeks postoperatively, the nm-HA/PPF implant had been completely resorbed and replaced with newly formed bone. At seven weeks, the cranial defect had essentially healed with nearly complete remodeling of the defect area when compared to the samples procured at 4 weeks postoperatively.

In Group C, demineralized bone matrix had had been implanted for control purposes. Histologic observations differed from the PPF-implanted groups (Groups A and B) in that the implanted demineralized bone matrix could not be clearly identified at any of the postoperative follow up time points. The material appeared to have been resorbed as early as one week postoperatively. However new bone formation was noted as early as 4 weeks and appeared more pronounced at 7 weeks postoperatively. Near complete healing of the cranial defect was noted at 7 weeks postoperatively. At seven weeks postoperatively, the entire cranial defect was filled with loosely packed newly formed bone.

In contrast, control defects, where no implant was placed, remained empty until four weeks postoperatively. Some reactive bone formation originating from the periphery of the drill hole defect was noted. At seven weeks postoperatively, control drill hole defects appeared similar to the 4 week samples and were not filled with newly formed bone.

This was supported by the histomorphometric analysis. By quantitative volume measures as expressed by the New Bone Volume Index, the experimental defects treated with nm-HA/PPF implants showed highest amount of new bone formation (mean 95±17 percent) compared with control defects (without implant) (p<0.02).

In maxillofacial and mandibular reconstruction, filling of bony voids with graft material remains challenging due to lack of structural support throughout the course of new bone regeneration. A bioresorbable bone repair material made from the unsaturated polyester poly(propylene glycol-co-fumaric acid) (PPF) and two different types of hydroxylapatite, crosslinked in the presence of either μm-sized, or nm-sized hydroxylapatite filler and effervescent foaming agents, was prepared. This bone repair material develops porosity in vivo by generating carbon dioxide during the reaction of citric acid and sodium bicarbonate, which are responsible for controlled pore generation and expansion with respective pore sizes of 100-1000 μm. Two, noncritical, 4-mm diameter, cortical defects were produced in the calvaria of 28-day old Sprague Dawley rats. In each animal, one defect was treated with one of the following materials: a PPF formulation with μm-sized hydroxylapatite, PPF with nm-sized hydroxylapatite, and demineralized bone matrix. The second defect was left to heal unaided to serve as paired control. Four sets of 24 animals each were evaluated at 1, 2, 4, and 7 weeks postoperatively including eight animals treated with each fill material. Radiographic and histologic techniques were employed to analyze the amount of new bone formation and the presence of inflammatory infiltrates at the repair site.

Histologic analysis of the healing process revealed superior healing of the cranial defects with both formulations of PPF bone repair material when compared to the control defects, which were left empty. Remodeling of the newly formed bone appeared more advanced with use of the PPF formulation containing nm-sized hydroxyapatite. These findings were corroborated by the histomorphometric analysis of new bone formation. Inflamatory cells were only noted in the one-week groups and were not related to material type. Inflamatory infiltrates were absent in all other groups evaluated at later postoperative times. Results of this study demonstrated both biocompatibility and osteoconductive properties of the porous PPF-based bone repair material in a cranial defect model.

### Example 7

### A Double Blinded Controlled Parallel Study of Four PPF/HA Formulations on Healing Mandibular Defects in Rats

The objective of this study was to evaluate the efficacy of alloplastic graft formulations administered topically to promote the healing of mandible defects created in rats.

Material and Methods

One hundred sixty (160) rats were randomized into five groups of thirty-two (32) animals: the positive control group and four (4) test groups. All groups were assigned a different treatment. On day 0, each animal's health status was checked. The animals were then weighed, randomized and numbered. Surgery was performed. Specimens were collected at the four time points (week 1, 2, 4 and 7).

Animals were weighed and then anesthetized with a single ip. injection of ketamine HCl (100 mg/kg) and xylazine (5 mg/kg). The mandibular rami was exposed bilaterally on the right and left side. In each animal, a mandibular defect was created in each side by drilling 4-mm holes using a rotary drill. Defects were irrigated with Ringer's lactate. In all animals, the left side defect was untreated. The right side defect of Group 1 were packed with demineralized human bone matrix commercially available as Grafton Putty® from the Musculoskeletal Transplant Foundation. The right side defect of Groups 2, 3, and 4 were packed with one of the three PPF-based formulations and Group 5 were packed with autograft only. Incisions were closed using interrupted sutures. The area were cleaned and covered with a wound healing liquid. The rats were given an ip. prophylactic dose of penicillin G (25,000 U/kg). One hour after surgery, each rat received an ip. dose of Buprenorphine (0.05 mg/kg).

Eight animals from each group were sacrificed at Week 1, 2, 4, and 7 postoperatively. Specimens were wrapped in saline-soaked gauze and shipped to the sponsor for evaluation.

Test article (cross-linked PPF containing HA particles organized as described above) was prepared by technicians from Cambridge Scientific, Inc. Test article was packed gently at a low viscosity (within 2 minutes of mixing) into the defects made in the right mandible. In the positive control group, demineralized human bone matrix (Grafton Putty® from the Musculoskeletal Transplant Foundation) was mixed to a thick slurry by combining the powder with saline, then packed into the defect with slight overfill. The defects were closed 5 minutes after placement of the test implant material. Incisions were closed with interrupted sutures.

Sectioned mandibles (2 per film) were placed exterior side up on Kodak Occusal film. Radiographs were. The left and right defect radiographs were blinded for treatment and measured along the x and y-axis. The mean of the x and y-axis measurements was calculated and total area calculated. Total area was graphed. Percent difference between the left and right defects was calculated and graphed.

Results

Mean mandible defect area for each test article was determined. Mean area of the left side defect (untreated control) and right side defect (test implant) were determined and showed significant differences between 100% Autograft and its control (p<0.001) at week 4 and 75% PPF/HA+25% Autograft (p=0.007) and its control at week 4.

The plot of the area for the untreated defect and defect receiving demineralized bone from the same mandible was calculated from the mean of the x and y axis using the area of a circle equation (πr²). Group means and standard errors of the mean (SEM) were calculated for each week. The plot shows the defect area for untreated and defect receiving demineralized bone.

The mandible defect area treated with a PPF/HA formulation was determined. Area for the untreated defect and defect receiving 100% PPF/HA from the same mandible was calculated from the mean of the x and y axis using the area of a circle equation (πr²). Group means and standard errors of the mean (SEM) were calculated for each week for untreated defect and defect receiving 100% PPF/HA. FIG. 5 compares the defect area for untreated defect and defect receiving 25%PPF/HA-75%Autograft. The defect area for untreated defect and defect receiving 100%Autograft. T-test showed that the untreated defect area was significantly smaller than the area of the defect implanted 100%Autograft (p<0.001) at week 4. The defect area for untreated defect and defect receiving 75%PPF/HA-25%Autograft was compared. T-test showed that the untreated defect area was significantly smaller than the area of the defect implanted 100%Autograft (p=0.007) at week 4. The results demonstrate that the untreated (negative) controls of the four test groups are statistically equivalent, except for demineralized bone and 100% PPF/HA at week 1. The results demonstrate that a mixture of PPF/HA with or without Autografts achieves healing at a rate similar to treatment with 100% Autograph.

### Example 8

### Repair of Bony Cranial Defects

A similar study to repair bony cranial defects was conducted.

Methods

In each animal, two cranial bony defects were created using an established animal model. Prior to surgery, animals were anesthetized by an ip. injection of ketamine HCl (100 mg/kg) and xylazine (5 mg/kg). The surgical site were shaved and scrubbed with a solution of Betadine (povidone-iodine) and alcohol (Dura-Prep; 3M Health Care, St. Paul, Md., USA). The calvarium was exposed by making a 3 cm longitudinal incision in the occipital cranium of the rat. The periosteum was stripped and two 4 mm diameter full thickness bony holes were created side by side by removal of a bone disc of similar size using a rotary drill and irrigated with lactated Ringer's solution. After hemostasis was achieved, implantation began. In all animals left side defect was untreated. Right side defect received implant. The soft tissues and skin were closed in layers with interrupted absorbable sutures. The rats were given a prophylactic ip. dose of penicillin G (25,000 U/kg). Buprenorphine (0.05 mg/kg) was administered intramuscularly one hour after surgery as an analgesic.

Results

The Following Results were Obtained:

The area of the defect filled with demineralized bone, week 1 is 8.2% smaller that of its control. The area of the defect filled with demineralized bone, week 2 is 0.7% larger than that of its control. The area of the defect filled with demineralized bone, week 4 is 56.1% smaller that of its control. The area of the defect filled with demineralized bone, week 7 is 117.8% smaller that of its control.

The area of the defect filled with PPF/μm HA, week 1 is 30.7% smaller that of its control. The area of the defect filled with PPF/μm HA, week 2 is 29.4% smaller that of its control. The area of the defect filled with PPF/μm HA, week 4 is 150.6% smaller than that of its control. The area of the defect filled with PPF/μm HA, week 7 is 307.4% larger than that of its control.

The area of the defect filled with PPF/nmHA, week 1 is 23.6% smaller than that of its control. The area of the defect filled with PPF/nm HA, week 2 is 30.5% smaller that of its control. The area of the defect filled with PPF/nm HA, week 4 is 123.0% smaller that its control. The area of the defect filled with PPF/nm HA, week 7 is 30.4% smaller that of its control. There was significant difference in defect area in defects filled with PPF/nmHA versus untreated defects at week 2 (p=0.004). There was significant difference in defect area between defects filled with PPF/nmHA versus demineralized bone at week 2 (p=0.014).

Those skilled in the art will recognize, or be able to ascertain using no more than routine experimentation, many equivalents to the specific embodiments of the present application described herein. Such equivalents are intended to be encompassed by the following claims.

We claim:

1. A bioresorbable osteoconductive composition for bone repair comprising:
a) a bioresorbable polymer
b) a micro or nano biocompatible filler; and
c) pores or a pore creating substance.

2. The composition of claim 1 wherein the filler is selected from the group consisting of metals, calcium carbonate, carbon, bioceramics, and synthetic materials.

3. The composition of claim 1 wherein the filler is a bioceramics.

4. The composition of claim 1 wherein the filler is hydroxyapatite.

5. The composition of claim 1 wherein the filler is a metal.

6. The composition of claim 5 wherein the bioresorbable polymer is selected from the group consisting of poly(L-lactic acid), poly(D L-lactic acid), poly(D L-lactic-co-glycolic acid), poly(glycolic acid), poly(epsilon-caprolactone), polyorthoesters, polyanhydrides, polydioxanone, copoly(ether-esters), polyamides polylactones, and polyesters formed of an acid selected from the group consisting of citric, isocitric, cis-aconitic, alpha-ketoglutaric, succinic, malic, oxaloacetic and fumaric acid, and combination thereof.

7. The composition of claim 6 wherein the bioresorbable polymer is a polyester which is cross-linkable with a cross-linking agent.

8. The composition of claim 7 wherein the bioresorbable polymer is poly(propylene glycol-fumaric acid).

9. The composition of claim 8 wherein the cross-linking agent is selected from the group consisting of vinyl pyrrolidone and methyl methacrylate.

10. The composition of claim 9 wherein the cross-linking agent is vinyl pyrrolidone.

11. The composition of claim 1 comprising pores in the range of between 100 and 1000 microns.

12. The composition of claim 1 comprising a pore forming agent, wherein the pore creating substance is an effervescent agent.

13. The composition of claim 12 wherein the effervescent agent is a carbonate and an acid.

14. The composition of claim 1 comprising a pore forming agent selected from the group consisting of particles leachable with a non-solvent for the polymer and volatile salts.

15. The composition of claim 1 comprising poly(propylene glycol-fumaric acid) and hyaluronic acid particles as a filler.

16. The composition of claim 1 further comprising a biologically active material.

17. The composition of claim 1 further comprising graft material selected from the group consisting of allograft, autograft, xenograft, and demineralized bone.

18. The composition of claim 16 wherein the biologically active material is selected from the group consisting of cells and therapeutic agents.

19. The composition of claim 18 wherein the biologically active material is a therapeutic agent selected from the group consisting of growth factors, antibiotics, antivirals, antifungals, immunostimulators, and immunosuppressants.

20. The composition of claim 1 wherein the composition is in the form of a scaffold.

21. The composition of claim 1 wherein the composition further comprises fibers or other structural supports.

22. The composition of claim 1 formed into an implant for implantation into a site for repair or regeneration of bone.

23. A method for bone repair or regeneration comprising:
providing a composition at a site in need thereof, wherein the composition
a) a bioresorbable polymer
b) a micro or nano biocompatible filler; and
c) pores or a pore creating substance.

24. The method of claim 23 wherein the composition is implanted at a site for periodontal repair or regeneration.

25. The method of claim 18 wherein the composition is implanted at a site for alveolar repair or regeneration.

26. The method of claim 18 wherein the composition is implanted at a site for maxillary repair or regeneration.

27. The method of claim 19 wherein the composition is implanted at a site for periodontal regeneration, wherein the implanting step comprises:
inserting a subperiosteal implant on the dental bone ridge of a mammal subject which further comprises:
a) making an incision in the tissue covering the bone ridge;
b) tunneling the tissue such that it separates from the bone ridge;
c) injecting a periodontal regeneration system under the tissue;
d) securing the two parts of the implant together;
e) suturing the incision; and
f) inserting a subperiosteal implant system.

28. The method of claim 27 comprising administering a bioactive agent with the composition, comprising applying a therapeutically effective amount of a growth factor directly to the periodontal regeneration system.

29. The method of claim 28 wherein the growth factor is one or more factors selected from the group consisting of platelet-derived growth factor in a form having two beta chain (PDGF-BB), platelet-derived growth factor in a form having an alpha and a beta chain (PDGF-AB), IGF-I; and TGF-beta.

30. The method of claim 23 wherein the composition is formed into a prostehtic implant prior to implantation.

31. The method of claim 23 wherein the composition forms a bone graft extender.

32. The method of claim 31 wherein the composition comprises graft material.
